(19) United States
(12) Plant Patent Application Publication
(10) Pub. No.: US 2015/0351300 P1
(43) Pub. Date: Dec. 3, 2015

(54) Dianthus Plant Named 'KLEDG13158'

(50) ;

(71) Applicant: Klemm+Sohn GmbH & Co. KG, Stuttgart (DE)

(72) Inventors: Nils Klemm, Stuttgart (DE); Martin Glawe, Stuttgart (DE)

(73) Assignee: Klemm+Sohn GmbH & Co. KG, Stuttgart (DE)

(21) Appl. No.: 14/120,505

(22) Filed: May 27, 2014

(51) Int. Cl. A01H 5/00

(52) U.S. Cl. PLT/281

(57) ABSTRACT

A new Dianthus plant particularly distinguished by single-type pink flowers with purple eyes, good branching, and an upright and bushy plant habit, is disclosed.

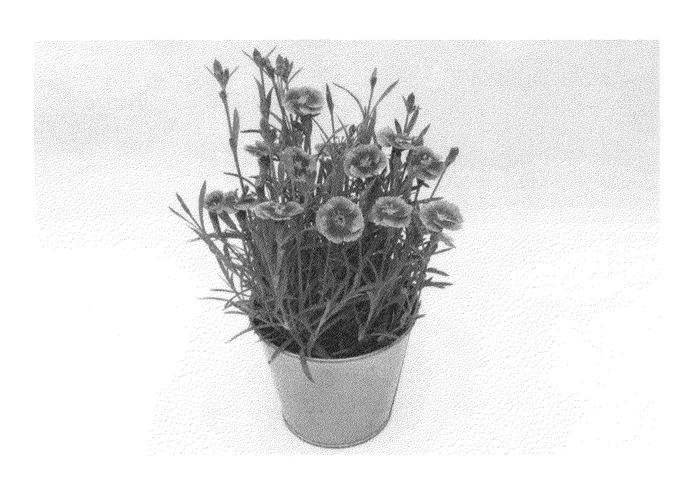

### GENUS AND SPECIES

Dianthus sp.

### VARIETY DENOMINATION

‘KLEDG13158’

### BACKGROUND OF THE NEW PLANT

The present invention comprises a new and distinct variety of Dianthus, botanically known as Dianthus sp., and hereinafter referred to by the variety name ‘KLEDG13158’. ‘KLEDG13158’ originated from a cross-pollination between the proprietary female Dianthus parent ‘DSS 2007 0051’ (unpatented) and proprietary male Dianthus parent ‘DSS 2008 0015’ (unpatented) in June 2009 in Stuttgart, Germany.

The seeds from the cross were sown and plants were grown in a greenhouse for evaluation, where an individual plant designated ‘KLEDG13158’ was selected from the group of plants June 2010 in Stuttgart, Germany. In June 2010, ‘KLEDG13158’ was first vegetatively propagated by vegetative cuttings. ‘KLEDG13158’ was found to reproduce true to type in successive generations of asexual propagation in Stuttgart, Germany via vegetative cuttings.

### SUMMARY

The following are the most outstanding and distinguishing characteristics of this new variety when grown under normal horticultural practices in Stuttgart, Germany.

1. Single-type pink flowers with purple eyes;

2. Good branching; and

3. An upright and bushy plant habit.

### CROSS-REFERENCE TO RELATED APPLICATIONS

A Community Plant Variety Rights application was filed with the European Community Plant Variety Rights Office on Jul. 1, 2013, Application No. 2013/1808; and a Plant Breeders Rights application was filed in Switzerland on May 31, 2013. ‘KLEDG13158’ has not been sold or made publicly available more than one year prior to the filing date of the present application.

### DESCRIPTION OF THE PHOTOGRAPH

This new Dianthus plant is illustrated by the accompanying photograph of a plant approximately 4 to 5-months old and grown in a glass greenhouse in Stuttgart, Germany. The photograph shows the whole plant, including the flowers and foliage and buds and was taken in April 2011 in Stuttgart, Germany. The colors shown are as true as can be reasonably obtained by conventional photographic procedures.

### DESCRIPTION OF THE NEW VARIETY

The following detailed descriptions set forth the distinctive characteristics of ‘KLEDG13158’. The data which define these characteristics were collected from asexual reproductions carried out in Stuttgart, Germany. Data was collected on 4-month old plants grown in a greenhouse in March 2014. Color references are to The R.H.S. Colour Chart of The Royal Horticultural Society of London (R.H.S.), 5^{th} edition (2007).
Classification:
   Family.—Caryophyllaceae.
   Botanical.—Dianthus sp.
   Common.—Garden carnation.
   Designation.—‘KLEDG13158’.
Parentage:
   Female parent.—The proprietary Dianthus variety ‘DSS 2007 0051’ (unpatented).
   Male parent.—The proprietary Dianthus variety ‘DSS 2008 0015’ (unpatented).
Plant:
   Propagation type.—Vegetative cuttings.
   Time to produce a rooted young plant.—20 days.
   Root description.—White, well-branched, and fine.
   Rooting habit.—Lanceolate.
   Plant form.—Potted and garden plant, upright and bushy.
   Plant height.—15.0 cm.
   Plant diameter or spread.—16.0 cm.
Lateral branches:
   Length.—6.0 cm.
   Diameter.—2.0 cm.
   Internode length.—1.3 cm.
   Node length.—0.5 cm.
   Node diameter.—0.4 cm.
   Texture.—Smooth.
   Color.—RHS 137D.
Foliage:
   Arrangement.—Opposite.
   Length.—5.0 cm.
   Width.—0.4 cm.
   Shape.—Lanceolate.
   Apex.—Acute.
   Base.—Obtuse.
   Margin.—Entire.
   Texture (both upper and lower surfaces).—Smooth.
   Immature leaf color.—Upper surface: RHS 139C Lower surface: RHS 139D.
   Mature leaf color.—Upper surface: RHS 139C Lower surface: RHS 139D.
   Venation pattern.—Pinnate.
   Venation color (both upper and lower surfaces).—RHS 139C.
Flowers:
   Flower type and habit.—Single.
   Fragrance.—Present.
   Natural flowering season.—Spring to fall.
   Flower longevity.—6 to 8 days.
   Buds.—Length: 1.4 cm Diameter: 0.5 cm Shape: Tall cylinder to ovate Color: RHS 187A.
   Corolla.—Quantity of petals and arrangement: 5 arranged radially Corolla height: 1.0 cm Petal length: 3.3 cm Petal width: 2.0 cm Petal shape: V-shaped Petal apex: Obtuse Petal base: Cuspidate Petal margin: Serrate Petal texture (both upper and lower surfaces): Smooth Petal color: Immature: Upper surface: RHS 60A and RHS 63B Lower surface: RHS N74C Mature: Upper surface: RHS 60A and RHS 63C Lower surface: RHS N74D.
   Calyx.—Quantity of sepals and arrangement: Composed of 5 sepals Calyx height: 1.5 cm Calyx diameter (at the apex): 0.5 cm Sepal texture (both upper and lower surfaces): Smooth Sepal color: Upper surface: RHS 187B Lower surface: RHS 177A.
Peduncles:
   Length.—10.0 cm.
   Diameter.—0.2 cm.
   Strength.—Moderate.
   Texture.—Smooth.
   Color.—RHS N138D.
Reproductive organs:
   Stamens.—Quantity per flower: 5 Filament length: 1.0 cm Filament color: RHS 69D Anther length: 0.2 cm Anther diameter: 0.08 cm Anther color: RHS 84D Pollen amount: Moderate.
   Pistils.—Quantity per flower: 2 Pistil length: 2.0 cm Style length: 0.1 cm Style color: RHS 73D Stigma shape: Filamentous Stigma color: RHS 59C Ovary size: 0.6 cm Ovary shape: Ovate Ovary color: RHS 139D.
Fruit and seed set: No fruit and seed set observed.
Disease and pest/insect resistance: No disease and pest/insect resistance observed.

### COMPARISON WITH PARENTAL LINES AND KNOWN VARIETY

‘KLEDG13158’ is a distinct variety of Dianthus and is distinguished from its parents as described in Table 1.

TABLE 1
Comparison with Parental Lines

| | | Female Parent | Male Parent |
|---|---|---|---|
| Characteristic | ‘KLEDG13158’ | ‘DSS 2007 0051’ | ‘DSS 2008 0015’ |
| Flower color | Pink with purple eye | Dark pink | Light pink |
| Branching | Very good | Medium | Medium |

‘KLEDG13158’ is a distinct variety of Dianthus and is most similar to the commercial Dianthus variety ‘KLEDG10117’ (U.S. Plant Pat. No. 23,330), also commercially known as Everlast™ ‘Lavender+Eye’. Differences between the varieties are described in Table 2.

TABLE 2
Comparison with Similar Variety

| Characteristic | ‘KLEDG13158’ | ‘KLEDG10117’ |
|---|---|---|
| Flower color | Pink with purple eye | Lavender with purple eye |
| Plant height | High | Medium |
| Flower type | Single | Double |

We claim:

1. A new and distinct variety of Dianthus plant designated ‘KLEDG13158’ as shown and described herein.
